(19) United States
(12) Patent Application Publication
(10) Pub. No.: US 2024/0157054 A1
(43) Pub. Date: May 16, 2024

(54) METHOD AND SYSTEM OF PENALIZATION FOR MODEL PREDICTIVE CONTROL IN AUTOMATED INSULIN DELIVERY

(71) Applicant: University of Virginia Patent Foundation, Charlottesville, VA (US)

(72) Inventors: Marcela MOSCOSO-VASQUEZ, Charlottesville, VA (US); Jose GARCIA-TIRADO, Bern (CH); Patricio COLMEGNA, Charlottesville, VA (US); Marc D. BRETON, Charlottesville, VA (US)

(21) Appl. No.: 18/507,253

(22) Filed: Nov. 13, 2023

Related U.S. Application Data
(60) Provisional application No. 63/424,639, filed on Nov. 11, 2022.

(51) Int. Cl. A61M 5/172; G16H 20/17

(52) U.S. Cl. CPC A61M 5/1723; G16H 20/17; A61M 2005/1726; A61M 2230/201

(57) ABSTRACT

A method, system, and computer-readable medium are provided for an Automated Insulin Delivery (AID) system in which Model Predictive Control (MPC) thereof implements weighting of glucose target error relative to corresponding predicted blood glucose (BG) levels according to insulin on board (IOB). Accordingly, basal insulin infusion and available additional bolusing each in connection with glycemic disturbances such as unannounced meals can be proximally administered to maintain time in range (TIR) without incurring insulin stacking.

### CROSS-REFERENCE TO RELATED APPLICATION

This international application claims priority to and the benefit of U.S. Provisional Application No. 63/424,639 filed Nov. 11, 2022, the entire contents of which are incorporated by reference herein.

### STATEMENT OF GOVERNMENT INTEREST

This invention was made with government support under Grant No. DK129553 awarded by The U.S. National Institutes of Health. The government has certain rights in the invention.

### FIELD OF THE DISCLOSURE

Disclosed embodiments relate to providing improved glycemic control to individuals with Type 1 diabetes mellitus (T1DM; herein T1D), and more specifically, such improvement as may be implemented in accordance with real-time adjustment for insulin dosing owing to continuing measurement for weighting of glucose target error comprising Model Predictive Control (MPC) of an Automated Insulin Delivery (AID) system that is then operable so as to fully automate the rejection of glycemic disturbances associated with, for example, absence of meal announcement.

### BACKGROUND

Herein, parenthetical references to citations provided throughout the forthcoming discussion(s) refer to those specifically mentioned documents listed in the section entitled “References” appearing at the conclusion of this document.

T1D is a chronic disease characterized by the autoimmune destruction of insulin-producing pancreatic β-cells. As a consequence, individuals with this condition require lifelong insulin replacement to regulate blood glucose (BG) concentration. Such regulation is delivered by either Multiple Daily Injections (MDI), a Sensor-Augmented Pump (SAP), or more recently AID systems. However, despite the progress in diabetes technology, still close to 80% of American adults fail to reach the recommended HbA1c target of less than 7% (Wolfsdorf and Ratner, 2019). Disturbances including meals and physical exercise remain the most challenging events for fully implemented AID systems given the delays in absorption and action of subcutaneously injected insulin. For instance and in the case of meals, there exists compromise between the controller's aggressiveness to counteract hyperglycemia and late postprandial hypoglycemia originated by insulin stacking (Colmegna et al., 2021).

MPC is recognized as one of the most popular control algorithms for AID systems available to manage T1D, having been tested in a variety of formulations (Camacho and Alba, 2013; Garcia-Tirado et al., 2021; Hovorka et al., 2004; Messori et al., 2016; Gondhalekar et al., 2018; Hajizadeh et al., 2019; Villa-Tamayo and Rivadeneira, 2020). Most of these contributions involve an adaptive capability that uses a dynamically updated system model and a MPC cost function with constant structure or parameters or a fixed system model (i.e., personalized or population-based) that varies the MPC cost function, or a combination of both (i.e., updated and fixed systems).

Different ways of adapting the MPC cost function have been studied in order to minimize that function, i.e., relative to the difference between predicted and targeted values. Villa-Tamayo and Rivadeneira (2020) defined four sets of controller parameters to regulate aggressiveness of the controller according to the risk of either hypo- or hyperglycemia, though prioritizing the latter. The MPC formulation proposed by Hajizadeh et al. (2019) employs glucose and insulin concentration risk indices to manipulate the penalty weights in the cost function. Here, set-point deviations are penalized based on an adaptive glycemic risk index that increases rapidly in response to hypoglycemic excursions, while such index is more gradual for hyperglycemic excursions. As a result, the aggressiveness of insulin dosing is penalized according to the insulin risk index so that the insulin infusion rate becomes suppressed if sufficient insulin is present in the bloodstream. Time-varying weights for penalizing deviations from the target were considered by Gondhalekar et al. (2018). Such a system includes an adaptive MPC cost function that is based on predicted glucose values and their rate of change to modulate the controller's aggressiveness at the start of hyperglycemia while causing the controller to be more conservative as glucose levels decrease.

All of these approaches have proven to be useful in various MPC formulations. However, the impact on the controller's achievable performance of such modifications (relative to constant weights) has not been directly assessed such that a relevant cost function for MPC can be optimally minimized to thus enable a maximal amount of time spent in euglycemia (i.e., normal glucose levels).

Herein, we aim to perform such analysis on our MPC-based AID system—RocketAP. To this end, we evaluate two approaches: one with a constant error weight Q, and another one with an adaptive error weight that depends on insulin on board (IOB), Q(IOB). Simulations are performed using the 100-adult cohort of the U.S. Food and Drug Administration (FDA)-accepted University of Virginia (UVA)/Padova T1D Simulator (Kovatchev et al., 2009; Visentin et al., 2018), considering unannounced meals with different absorption rates.

### SUMMARY

It is to be understood that both the following summary and the detailed description are exemplary and explanatory and are intended to provide further explanation of the present embodiments as claimed. Neither the summary nor the description that follows is intended to define or limit the scope of the present embodiments to the particular features mentioned in the summary or in the description. Rather, the scope of the present embodiments is defined by the appended claims.

As may be understood from a review of the entireties of the discussions herein, disclosed embodiments evidence, as a result of simulation on the 100-adult cohort of the FDA-accepted UVA/Padova simulator (Kovatchev et al., 2009; Visentin et al., 2018), successfully adjusting MPC for an AID system to, both with and without delivery of insulin bolusing in response to disturbances such as unannounced meals, achieve advantageous time in range (TIR), i.e., (TIR, 70<BG<180 mg/dL). In particular, it will be appreciated that such embodiments employ modification, when attaining TIR, for weighting of the MPC's targeted error to modulate basal insulin infusion by accounting for a subject's insulin on board (IOB). In view of this accounting, the MPC can further cause the administration of one or more measured boluses that, in accordance with scheduling for administration of a total daily insulin (TDI) amount, counteract instance of hyperglycemia resulting from, for example, an unannounced meal event. In other words, as will be appreciated from discussion herein, IOB can be an invaluable measure by which to advantageously regulate MPC to optimize deterrence of both hypoglycemia and hyperglycemia.

An embodiment may provide, in an automated insulin delivery (AID) system, a processor-implemented method of regulating glycemia for a subject having Type 1 diabetes (T1D), including predicting blood glucose (BG) levels for the subject based on operation of a model predictive control (MPC) regime on continuous glucose monitor (CGM) measurements of the subject, wherein the MPC regime weights a glucose target error thereof according to a predetermined weighting factor Q_{z} in dependence on insulin on board (IOB) of the subject. The method may further include delivering basal insulin dosing to the subject according to the predicting to maintain real-time glycemia of the subject with a range of 70 mg/dL to 180 mg/dL.

For the weighted glucose target error, Q_{z} may weight a difference between the predicted BG levels for the subject and a reference BG level corresponding to the MPC regime.

Q_{z} may be adjusted based at least on a minimum amount of the IOB of the subject (IOBₘᵢₙ) in which IOBₘᵢₙ comprises a fraction of total daily insulin (TDI) required by the subject before Q_{z} can be decreased.

In response to detection of an elevation in one or more of the CGM measurements, a controller of the AID system increases basal insulin infusion to the subject to obtain a minimum amount of the IOB of the subject (IOBₘᵢₙ) in which IOBₘᵢₙ comprises a fraction of total daily insulin (TDI) required by the subject before Q_{z} can be decreased.

In response to detection of the elevation in one or more of the CGM measurements, the controller of the AID system supplements the IOBₘᵢₙ with an insulin bolus responsive to the elevation to effect the real-time glycemia of the subject to be between the range of 70 mg/dL to 180 mg/dL.

The insulin bolus is measured according to a percentage of the TDI (P(TDI), based on a predetermined probability (πₖ) that the elevation in one or more of the CGM measurements resulted from a glycemic disturbance comprising at least an unannounced meal.

The P(TDI) is given by the following schedule, in which:

$$P ( TDI ) = \{ \begin{matrix}0 \% if {π}_{k} < 0 \% \\ 7 \% if {π}_{k} \geq 30 \% \\ 10 \% if {π}_{k} \geq 40 \%\end{matrix} .$$

As the IOB of the subject decreases, Q_{z} is increased to cause the controller of the AID system to increase the basal insulin infusion to the subject to obtain the real-time glycemia of the subject to be between the range of 70 mg/dL to 180 mg/dL.

Respective embodiments may further include a relative system and computer-readable medium commensurate with the embodied method above.

In certain embodiments, the disclosed embodiments may include one or more of the features described herein.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. Embodiments herein will be more particularly described in conjunction with the following drawings wherein:

FIG. 1 illustrates an automated insulin delivery (AID) system implementing model predictive control (MPC) and a bolus priming system (BPS), according to embodiments herein;

FIG. 2 illustrates variation in an error weight (Q_{z}) implemented according to the MPC of FIG. 1, wherein the variation is depicted for differing levels of total daily insulin (TDI) relative to differing levels of minimum insulin on board (IOB), according to embodiments herein;

FIG. 3A illustrates, for an in-silico subject of the 100-adult cohort of the U.S. Food and Drug Administration (FDA)-accepted University of Virginia (UVA)/Padova simulator coordinated with the MPC according to embodiments herein, closed-loop control (CLC) response to breakfast relative to a first scenario for the simulation that is evaluated for blood glucose (BG);

FIG. 3B illustrates, for an in-silico subject of the 100-adult cohort of the UVA/Padova simulator coordinated with the MPC according to embodiments herein, closed-loop control (CLC) response to breakfast relative to the first scenario for the simulation that is evaluated for basal infusion;

FIG. 3C illustrates, for an in-silico subject of the 100-adult cohort of the UVA/Padova simulator coordinated with the MPC according to embodiments herein, (CLC) response to breakfast relative to the first scenario for the simulation that is evaluated for bolus priming system (BPS) boluses;

FIG. 3D illustrates, for an in-silico subject of the 100-adult cohort of the UVA/Padova simulator coordinated with the MPC according to embodiments herein, closed-loop control (CLC) response relative to the first scenario for the simulation that is evaluated for Q, variation;

FIG. 4 illustrates, for the MPC according to embodiments herein and relative to the 100-adult cohort of the UVA/Padova simulator, glycemic outcome metrics including percentage of insulin infused during the first and second hours, PP_{H1} and PP_{H2}, respectively after lunch relative to the first scenario of the simulation evaluated in FIGS. 3a-3d;

FIG. 5A illustrates, for an in-silico subject of the 100-adult cohort of the U.S. Food and Drug Administration (FDA)-accepted University of Virginia (UVA)/Padova simulator coordinated with the MPC according to embodiments herein, closed-loop control (CLC) response to breakfast relative to a second scenario for the simulation that is evaluated for blood glucose (BG);

FIG. 5B illustrates, for an in-silico subject of the 100-adult cohort of the UVA/Padova simulator coordinated with the MPC according to embodiments herein, closed-loop control (CLC) response to breakfast relative to the second scenario for the simulation that is evaluated for basal infusion;

FIG. 5C illustrates, for an in-silico subject of the 100-adult cohort of the UVA/Padova simulator coordinated with the MPC according to embodiments herein, closed-loop control (CLC) response relative to the second scenario for the simulation that is evaluated for bolus priming system (BPS) boluses;

FIG. 5D illustrates, for an in-silico subject of the 100-adult cohort of the UVA/Padova simulator coordinated with the MPC according to embodiments herein, closed-loop control (CLC) response to breakfast relative to the second scenario for the simulation that is evaluated for Q, variation;

FIG. 6 illustrates, for the MPC according to embodiments herein and relative to the 100-adult cohort of the UVA/Padova simulator, glycemic outcome metrics including percentage of insulin infused during the first and second hours, PP_{H1} and PP_{H2}, respectively after lunch relative to the second scenario of the simulation evaluated in FIGS. 5a-5d;

FIG. 7 illustrates an exemplary construct for the AID system of embodiments herein;

FIG. 8A illustrates an exemplary computing device which may implement one or more portions of the AID system of embodiments herein, and FIG. 8B illustrates a network system which may implement and/or be used in the implementation of one or more portions of the AID system of embodiments herein;

FIG. 9 illustrates a block diagram which may implement and/or be used in the implementation of one or more portions of the AID system herein in association with a connection to the Internet;

FIG. 10 illustrates a system which may implement and/or be used in the implementation of one or more portions of the AID system herein in accordance with one or more of a clinical setting and a connection to the Internet; and

FIG. 11 illustrates an exemplary architecture embodying one or more portions of the AID system herein.

### DETAILED DESCRIPTION

The present disclosure will now be described in terms of various exemplary embodiments. This specification discloses one or more embodiments that incorporate features of the present embodiments. The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. The skilled artisan will appreciate that a particular feature, structure, or characteristic described in connection with one embodiment is not necessarily limited to that embodiment but typically has relevance and applicability to one or more other embodiments.

In the several figures, like reference numerals may be used for like elements having like functions even in different drawings. The embodiments described, and their detailed construction and elements, are merely provided to assist in a comprehensive understanding of the present embodiments. Thus, it is apparent that the present embodiments may be carried out in a variety of ways, and does not require any of the specific features described herein. Also, well-known functions or constructions are not described in detail since they would obscure the present embodiments with unnecessary detail.

The description is not to be taken in a limiting sense, but is made merely for the purpose of illustrating the general principles of the present embodiments, since the scope of the present embodiments are best defined by the appended claims.

It should also be noted that in some alternative implementations, the blocks in a flowchart, the communications in a sequence-diagram, the states in a state-diagram, etc., may occur out of the orders illustrated in the figures. That is, the illustrated orders of the blocks/communications/states are not intended to be limiting. Rather, the illustrated blocks/communications/states may be reordered into any suitable order, and some of the blocks/communications/states could occur simultaneously.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedure, Section 2111.03.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. The word “exemplary” is used herein to mean “serving as an example, instance, or illustration.” Any embodiment described herein as “exemplary” is not necessarily to be construed as preferred or advantageous over other embodiments. Additionally, all embodiments described herein should be considered exemplary unless otherwise stated.

It should be appreciated that any of the components or modules referred to with regards to any of the embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Ranges may be expressed herein as from “about” or “approximately” one particular value and/or to “about” or “approximately” another particular value. When such a range is expressed, other exemplary embodiments include from the one particular value and/or to the other particular value.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. All references cited and discussed in this specification are incorporated herein by reference in their entireties and to the same extent as if each reference was individually incorporated by reference.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

In an effort to maximize percentage of time in range (TIR), i.e., (TIR, 70<BG<180 mg/dL), we, the inventors at the University of Virginia (UVA) present modification for a closed-loop control (CLC) automated insulin delivery (AID) system termed the Reactive Optimal Carbohydrates Kinetics EsTimation Artificial Pancreas (AP), or ROCKET AP (hereinafter “Rocket AP”), as more fully described in U.S. patent application Ser. No. 18/031,976 and WIPO Publication No. WO 2022/081788, each entitled, “METHOD AND SYSTEM OF CLOSED LOOP CONTROL IMPROVING GLYCEMIC RESPONSE FOLLOWING AN UNANNOUNCED SOURCE OF GLYCEMIC FLUCTUATION,” and which are commonly owned by the assignee of the present application and incorporated by reference herein. In particular, such modification is focused on (a) adjustment of the Rocket AP's model predictive control (MPC) to adapt the cost function thereof in accordance with a subject's insulin on board (IOB) in order to regulate basal insulin infusion toward time in range (TIR), i.e., (TIR, 70<BG<180 mg/dL), and alongside, (b) to adapt non-basal insulin bolusing in response to disturbances such as unannounced meals in accordance with a percentage of total daily insulin (TDI) amount (calculated by, for example, units=weight in pounds/4).

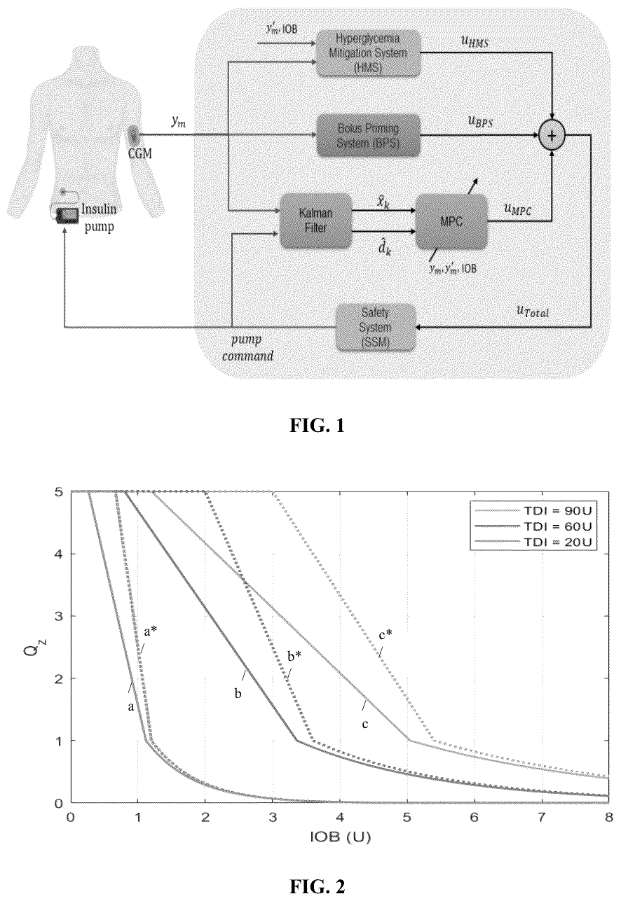

In these regards, and in referring to FIG. 1, the fully automated Rocket AP, as is explained in the aforementioned commonly owned documents and additionally in Garcia-Tirado et al. (2021), entails four main modules, including (i) a safety system (SSM) to compensate for imminent hypoglycemia by attenuating the control action, (ii) a Hyperglycemia Mitigation System (HMS) to compensate for prevailing hyperglycemia by delivering correction doses, (iii) a Bolus Priming System (BPS) to mitigate abrupt positive disturbances, and (iv) an MPC algorithm that regulates background insulin. Therein, and relative to the illustrated continuous glucose monitor (CGM) and insulin pump, yₘ, y′, and IOB represent current CGM measurement, its time derivative, and insulin on board, respectively, and whereas \hat{x}ₖ and [symbol] are the estimated states and disturbance at time k. Aspects including u_{HMS}, u_{BPS}, u_{MPC}, and U_{Total} represent relevant bolusing for the HMS, bolusing for the BPS, basal insulin infusion and total insulin as may be relevant to discussion herein. In connection with the MPC shown in FIG. 1, the same may be executed in accordance with the following MPC regime including equations (1a) through (1g), in which:

$$\begin{matrix}{min}_{{\tilde{u}}_{k} , {\tilde{η}}_{k}} {Φ}^{mpc} & ( 1 a ) \\ s . t . {x}_{k + j + 1 \vert k} = {Ax}_{k + j \vert k} + {B}_{I} {u}_{k + j \vert k} + {B}_{W} {w}_{k + j \vert k} & ( 1 b ) \\ {y}_{k} = {Cx}_{k} ∀ j = 1 , … , {N}_{p} & ( 1 c ) \\ {u}_{min} \leq {u}_{k + j \vert k} \leq {u}_{max} ∀ j ∈ [ 1 , … , {N}_{c} - 1 ] & ( 1 d ) \\ Δ {u}_{min} \leq Δ {u}_{k + j \vert k} \leq Δ {u}_{max} ∀ j ∈ [ 1 , … , {N}_{c} - 1 ] & ( 1 e ) \\ {y}_{min} - {y}_{k} \leq {η}_{k} & ( 1 f ) \\ {η}_{k} \geq 0 & ( 1 g )\end{matrix}$$

where Equation (1c) corresponds to the state-space representation of the prediction model that is obtained after linearizing and discretizing a modified Subcutaneous Oral Glucose Minimal Model (SOGMM) as described in Garcia-Tirado et al. (2021. Nₚ and N_{c} represent the prediction and control horizons, respectively, and whereas ũ=[u₁ u₂ . . . u_{N}_{_{c}}₋₁] and \tilde{η}=[η₁ η₂ . . . . η_{N}_{ₚ}₋₁] represent the control policy and a policy of slack variables, each of which is used to soften the hypoglycemia restraint provided by Equation (1f). The cost function in Equation (1a) for which minimization is targeted is given by the following, in which:

$${Φ}^{mpc} = {\sum }_{j = 0}^{{N}_{p}} {Q}_{z} {\overline{y}}_{k + j \vert k}^{2} + {κη}_{k + j \vert k}^{2} + {\sum }_{j = 0}^{{N}_{c} - 1} {λ}_{1} ( {y}_{m} , {y}_{m}^{′} ) Δ {u}_{k + j \vert k}^{2} , where {\overline{y}}_{k + j \vert k} = {y}_{k + j \vert k} - {r}_{k + j \vert k}$$

represents the glucose target error at the j-th step, r_{k+j|k} represents an asymmetric time-varying exponential reference signal as defined in Boiroux et al. (2017) and Garcia-Tirado et al. (2021), and k represents a constant penalizing predictions trending towards hypoglycemia. As such, Q_{z} may be considered to weight the glucose target error and, in consideration thereof, Q_{z} weights the difference between model prediction \tilde{y} of target BG levels and the evolution of the controller's reference \tilde{r} so as to penalize deviation from 70<BG<180 mg/dL. According to this framework, λ₁ is designed as a piece-wise function of the current BG value and its rate of change so as to allow more aggressive controller actions in response to high and rapidly changing BG values.

As will be understood from the discussion hereinafter, the AID system according to the Rocket AP further implements the BPS to further address (i.e., in addition to basal insulin infusion as modified herein) detected disturbances in BG levels resulting from instances of, for example, unannounced meals. In this regard, the BPS, as detailed in the aforementioned commonly owned documents incorporated herein, infuses increasing percentages of TDI as an estimated probability (πₖ) of a significant positive disturbance attains pre-specified thresholds. Here, such probability can be evaluated periodically, e.g., every five (5) minutes for the CGM trace to determine whether such a disturbance has occurred within the past 30 minutes. For one or more of such disturbances, TDI percentage dosages (P(TDI)) at each πₖ threshold can be computed according to a pre-determined schedule given as follows:

$$P ( TDI ) = \{ \begin{matrix}0 \% if {π}_{k} < 0 \% \\ 7 \% if {π}_{k} \geq 30 \% \\ 10 \% if {π}_{k} \geq 40 \%\end{matrix} ,$$

where, for the CGM trace, πₖ can be given by

$${π}_{k} = \frac{1}{1 + {e}^{- {y}_{log}}} .$$

Sizing of a particular bolus that can be automatically infused by the BPS of the Rocket AP is determinable according to an amount of IOB in consideration of, for example, BPS boluses previously infused according to the BPS and a scaling factor BPS_{sf}, and may be given by:

$${J}_{BPS} = {BPS}_{sf} max ( {P}_{TDI} TDI - \frac{{IOB}_{BPS}}{TDI} , 0 ) .$$

Toward the goal of minimizing the MPC's cost function and thus optimizing the MPC's prediction of BG, we, the inventors at the University of Virginia, studied varying tuning (i.e., adjusting) of the MPC's controller relative to manipulation of the error weight Q_{z} as either a constant value or one which is a function of IOB given that IOB is a widely used construct recognized to prevent insulin stacking, i.e., repeated insulin injection leading to overdosing due to delay in insulin action (Swan et al., 2009). Differently than the tuning/detuning rule based on IOB in Garcia-Tirado et al. (2019), such study as is discussed herein as regards Q_{z} based on IOB, i.e., Q_{z}(IOB), suggests modifying Q_{z} according to tiered levels of IOB as shown below, in which:

$${Q}_{z} ( IOB ) = \{ \begin{matrix}{Q}_{0} & if IOB < {IOB}_{min} \\ m \cdot IOB + {Q}_{0} & if IOB ∈ [ {IOB}_{min} , {IOB}_{thr} ] \\ 1 + {k}_{Q} [ 1 - {e}^{( {IOB}_{d} / {τ}_{Q} )} ] & if IOB > {IOB}_{thr}\end{matrix} ,$$

where Q₀ represents the nominal weight for Q_{z}(IOB),

$${IOB}_{min} = ϵ \frac{TDI}{α}$$

represents the allowed insulin threshold prior to Q_{z} being detuned (i.e., decreased) in any case, according to TDI, IOBₜₕᵣ represents the IOB concentration that would yield a value of Q_{z}=1, and α, ϵ and β are tunable parameters. The slope of the detuning rule relevant to operation of the MPC may be given as the following, in which:

$$m = \frac{{Q}_{0} \cdot α \cdot ( 1 - β )}{β \cdot ( TDI - α \cdot {IOB}_{min} )} .$$

As such, Q_{z} may be detuned according to how active insulin in a subject's body compares to that subject's specific daily insulin requirements, i.e., TDI. Accordingly, IOB daily (IOB_{d})=IOB−IOBₜₕᵣ, kQ is the maximum change on Q_{z} from Q_{z}=1 to Q_{z,min}=Q₀/β that guarantees continuity of Q_{z}, and τ_{Q} is the time constant regulating the first-order decay of Q_{z} and defined as follows:

$${τ}_{Q} = \frac{3 TDI - α {IOB}_{thr}}{5 α} ; {k}_{Q} = \frac{( {Q}_{0} / β ) - 1}{1 - {e}^{( {IOB}_{d} / {τ}_{Q} )}} .$$

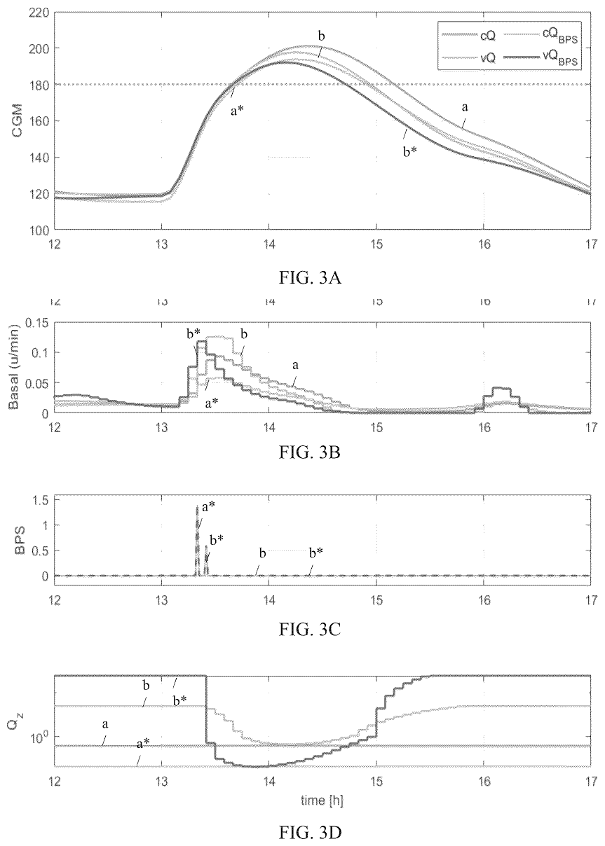

In referring to FIG. 2, wherein there is illustrated variation in an error weight Q_{z} implemented according to the MPC of FIG. 1 for differing levels of total daily insulin (TDI) relative to differing levels of minimum insulin on board (IOB), lines a and a* regard such variation of Q_{z} at TDI=20U and IOB==0.2TDI/α and at TDI=20U and IOBₘᵢₙ=0.5TDI/α, respectively, lines b and b* regard such variation of Q_{z} at TDI=60U and IOBₘᵢₙ=0.2TDI/α and at TDI=60U and IOB==0.5TDI/α, respectively, and lines c and c* regard such variation of Q_{z} at TDI=90U and IOBₘᵢₙ=0.2TDI/α and at TDI=90U and IOBₘᵢₙ=0.5TDI/α. This definition of Q_{z}, i.e., Q_{z}(IOB), demonstrates the MPC's controller ability to take an aggressive initial action upon detection of a disturbance and until sufficient insulin is infused (i.e., IOBₘᵢₙ). Q_{z} is then detuned with the linear segment, making it less sensitive to error, so as to allow the infused insulin to have an impact on glucose concentration (given the delay of the insulin absorption) and prevent insulin stacking. The addition of the exponential segment makes it possible to decrease Q_{z} quickly, though smoothly, until reaching the minimal Q₀/β value. In this way, abrupt increases in Q_{z} that could cause chattering in the control action are avoided and progressive re-tuning (i.e., increasing) of Q_{z} is permitted as insulin starts to clear. For instance, as IOB of the subject decreases, Q_{z} may be increased to cause a controller of the AID system herein to increase basal insulin infusion to the subject to obtain CGM measurements of the subject to be between the range of 70 mg/dL to 180 mg/dL. Advantageously, therefor, since tuning of Q_{z} depends on the user's TDI relative to IOB, such an adaptation of Q_{z} according to IOB enables the MPC to be tailored to an individual subject. In this way, Q_{z} represents a predetermined weighting factor in dependence of IOB of the subject and that may be adjusted based at least on a minimum amount of IOB of the subject in which such amount is based on a fraction of TDI required by the subject.

Relative to the tiered adaptation of Q_{z}, as discussed above, the following tuning parameters for the MPC's controller are selected as follows: Q_{z}′ initial value (Q0), the slope of the linear detuning segment (α) and its hysteresis (ϵ). Accordingly, an optimal set of parameters for gauging performance of the MPC's controller and BPS discussed herein (as against a constant Q_{z} and a Q_{z}(IOB), may include θ_{MPC}={Q₀, α, ϵ, BPS_{sf}} with respect to two separate pairs of configurations including (a) constant Q_{z} alone as cQ, (b) constant Q_{z} as cQ_{BPS} (indicating complement by the BPS), (c) Q_{z}(IOB) alone as vQ, and (d) vQ(IOB)_{BPS} (indicating complement by the BPS). Others of remaining controller parameters are provided in Table 1 below.

TABLE 1
Controller parameters

| Parameter | Value | | Parameter | Value | |
|---|---|---|---|---|---|
| Nₚ | 24 | | N_{c} | 18 | |
| uₘᵢₙ | −u_{b} | [mU/min] | uₘₐₓ | 200 | [mU/min] |
| κ | 100 | | Δuₘₐₓ | 50 | [mU/min] |
| τᵣ⁺ | 10 | [min] | yₘᵢₙ | 70 | [mg/dL] |

| | | | |
|---|---|---|---|
| λ_{1, nom} | 5/u_{b} | TDI | user-specific [U] |
| β | 1000 | | |

The parameter vector θ_{MPC} may be found by solving an optimization problem that minimizes the weighted sum of the risks of hypoglycemia (LBGI) and hyperglycemia (HBGI) (Kovatchev et al., 1997) and the number of hypoglycemic treatments per day. In this way, the performance index J_{Perf} may be given as:

$${J}_{Perf} = \frac{1}{100} {\sum }_{j = 1}^{100} [ \frac{1}{{N}_{Days}} {\sum }_{k = 1}^{{N}_{Days}} ( {HBGI}_{k}^{180} + 2 {LGBI}_{k}^{70} + {N}_{k , HT} ) ] ,$$

where N_{Days} represents the number of days considered in the optimization, N_{k,HT} represents the number of hypoglycemia treatments that were administered to the j^{th} subject on the k-th day, and HBGI¹⁸⁰ and LBGI⁷⁰ represent modified high and low blood glycemic indices based on the HBGI and LBGI as found in Kovatchev et al. (1997), respectively, in which such modification was introduced to emphasize TIR while penalizing deviations therefrom and is given by:

$${Risk}_{n} = {log ( {BG}_{k} )}^{1.084} - 5.381 \\ {LBGI}_{k}^{70} = {\sum }_{n = 1}^{{N}_{s}} 22.7 {Risk}_{n}^{2} ∀ {Risk}_{n} \leq 0 and {BG}_{k} < 70 mg / dL \\ {HBGI}_{k}^{180} = {\sum }_{n = 1}^{{N}_{s}} 22.7 {Risk}_{n}^{2} ∀ {Risk}_{n} > 0 and {BG}_{k} > 180 mg / dL .$$

MPC optimization was carried out via grid-search considering the values and intervals for the parameters shown in Table 2 below.

TABLE 2
Search space for tunning parameters

| | | |
|---|---|---|
| Parameter | Range | No. of values |

| | | | |
|---|---|---|---|
| Q₀ | constant Q_{z} | [0.1, 35] | 14 |
| | Q_{z}(IOB) | [1, 35] | 9 |
| α | | [2.5, 30] | 7 |
| ϵ | | [0, 0.5] | 6 |

| | | |
|---|---|---|
| BPS_{factor} | [0.2, 1.4] | 7 |

For each combination of Q_{z}-BPS, the relevant parameters were selected to form the grid. Table 3 below presents the optimal cases for completion of the parameter vector θ_{MPC}={Q₀, α, ϵ, BPS_{sf}} with respect to the four configurations including (a) constant Q_{z} alone as cQ, (b) constant Q_{z} as cQ_{BPS} (indicating complement by the BPS), (c) Q_{z}(IOB) alone as vQ, and (d) vQ(IOB)_{BPS} (indicating complement by the BPS), selecting them by their optimal J_{Perf}.

TABLE 3
Controller tuning parameters

| | | | | | |
|---|---|---|---|---|---|
| Configuration | Q_{z} | α | ϵ | BPS_{sf} | J_{Perf} |

Optimal

| | | | | | |
|---|---|---|---|---|---|
| cQ | 4.5 | — | — | — | 4.17 |
| cQ_{BPS} | 3.5 | — | — | 0.6 | 3.9 |
| vQ | 35 | 10 | 0.4 | — | 3.71 |
| vQ_{BPS} | 25 | 15 | 0.5 | 0.6 | 3.5 |

Fixing hypoglycemia exposure

| | | | | | |
|---|---|---|---|---|---|
| cQ | 0.6 | — | — | — | 4.85 |
| cQ_{BPS} | 0.2 | — | — | 0.6 | 4.86 |
| vQ | 5 | 10 | 0.3 | — | 4.01 |
| vQ_{BPS} | 25 | 15 | 0.5 | 0.6 | 3.5 |

To validate performance of the MPC of the Rocket AP toward demonstrating that varying the error weight Q_{z} as a function of IOB delivers improved TIR than does Q_{z} when maintained as a constant value, simulations according to Tables 2 and 3 were conducted for the 100-adult cohort of the UVA/Padova simulator. In particular, the simulations entailed separate 48-hour scenarios designed with unannounced meals having fixed time and contents, and considering (i) fast-absorbing meals with no snacks and (ii) mixed (slow-absorbing and fast-absorbing) meals with snacks. Such scenarios are detailed below.

### Scenario 1

In this first scenario, three meals were provided every day at 7:00 h, 13:00 h and 19:00 h, with carbohydrate content of 0.77, 0.77 and 0.65 g/kg for day one and 0.85, 0.70 and 0.9 g/kg for day two, respectively. Intra-day variability in insulin sensitivity and dawn phenomenon were included. 15-g hypoglycemia treatments were administered for BG<60 mg/dL, waiting 15 minutes before administering a new treatment. As reference, this scenario was selected when choosing the MPC's controller tuning parameters, as have been previously discussed.

According to the values of J_{Perf} presented in Table 3, it is demonstrated that better performance (i.e., at least better TIR) is achieved using Q_{z}(IOB), and also when combined with functionality for the BPS, i.e., vQ_{BPS}. Considering that the performance of a closed-loop system is analyzed with more than one metric (hypo- and hyperglycemia exposure, for instance), the comparison for the different configurations (a)-(b) is complicated given the interplay of these measures. Therefore, hereinafter comparison between all Q_{z}-BPS configurations is made by setting the exposure condition to hypoglycemia (both time below range (TBR) (i.e., <70 mg/dL and hypoglycemia treatments) throughout all cases to that of the optimal case, i.e., vQ_{BPS}, and evaluating the achievable performance under this situation. Under this condition, the optimal case was still observed in accordance with vQ_{BPS} (lower J_{Perf}) configuration, such that increased TIR and, consequently, lower time above range (TAR), i.e., >250 mg/dL) result.

In these regards, glycemic outcome metrics for the 100-adult population are presented in Table 4 below.

TABLE 4
Glycemic Outcome metrics for the in-silico scenarios.

| | Percentage of time in range (%) | | | | | No. |
|---|---|---|---|---|---|---|
| | BG < 54 | BG < 70 | 70 < BG < | BG > 180 | BG > 250 | Hypoglyc. |
| Configuration | mg/dL | mg/dL | 180 mg/dL | mg/dL | mg/dL | treatments |

Scenario 1

| | | | | | | | | | | | |
|---|---|---|---|---|---|---|---|---|---|---|---|
| cQ | 0 | [0 0.1] | 0 | [0 2.1] | 71.1 | [64 76.8] | 28.9 | [23.1 35.5] | 0 | [0 5.4] | 0 [0 1] |
| cQ_{BPS} | 0 | [0 0.2] | 0 | [0 2.1] | 71.8 | [64.2 76.5] | 27.5 | [23 35.4] | 0 | [0 5.6] | 0 [0 1] |
| vQ | 0 | [0 0.3] | 0 | [0 2.1] | 75 | [68.5 81.7] | 23.5 | [17.3 30.9] | 0 | [0 2] | 0 [0 1] |
| vQ_{BPS} | 0 | [0 0.3] | 0 | [0 2.1] | 79.5 | [70.7 84.8] | 20 | [13.9 28.3] | 0 | [0 0] | 0 [0 1] |

Scenario 2

| | | | | | | | | | | | |
|---|---|---|---|---|---|---|---|---|---|---|---|
| cQ | 0 | [0 0] | 0 | [0 0] | 67.4 | [58 74.9] | 32.6 | [25.1 42] | 0.8666 | [0 7.9] | 0 [0 0] |
| cQ_{BPS} | 0 | [0 0] | 0 | [0 0] | 76.2 | [65.8 86.6] | 23 | [12.8 33.2] | 0 | [0 1.4] | 0 [0 0] |
| vQ | 0 | [0 0] | 0 | [0 0.7] | 78.2 | [68.7 88.6] | 21.2 | [10.7 30.2] | 0 | [0 0.4] | 0 [0 0] |

| | | | | | | | | | |
|---|---|---|---|---|---|---|---|---|---|
| vQ_{BPS} | [0] | 0 | [0 0] | 0 | [0 0] | 0 | [0 0] | [0] | [0] |

* Outcome metrics are reported as median [25^{th} 75^{th}] percentiles.

Considering the same TBR and the number of hypotreatments, better performance (higher TIR and lower TAR) is observed when Q_{z} is a function of IOB, i.e., with configurations vQ and vQ_{BPS} than can be obtained in accordance with configurations cQ and cQ_{BPS}, in which such latter configurations fix Q_{z} as a constant value. Moreover, the effect of BPS can be observed by comparing either cQ with cQ_{BPS} or vQ with vQ_{BPS}. For example, complementing configuration cQ, in which Q_{z} is constant, with functionality of the BPS provides no significant overall performance as to TIR. In contrast, a 6% increase in TIR is obtained when using configuration vQ_{BPS} when compared to configuration vQ in which Q_{z} is solely a function of IOB.

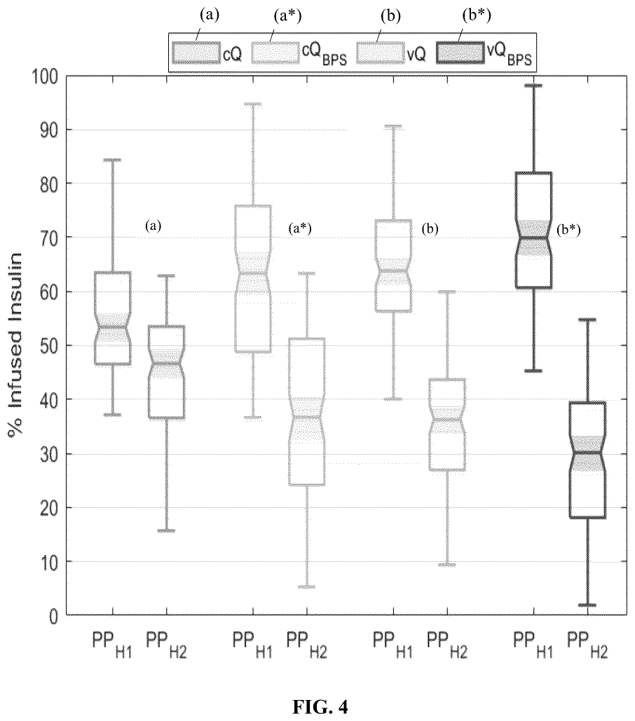

In referring to FIGS. 3(a)-3(d) detailing CGM trace, basal insulin infusion, BPS bolusing, and Q_{z} variation for the closed-loop response for a subject according to the four controller configurations discussed herein with regard to breakfast during the study, lines a and a* represent configurations cQ and cQ_{BPS} and lines b and b* represent configurations vQ and vQ_{BPS}, respectively. As such, the postprandial excursion observed with all configurations to lunch is presented for a representative subject. As shown, a lower peak in CGM measurement during the meal response is obtained with vQ_{BPS}, which also presents an elevated insulin delivery towards the start of the meal response when compared to others of the configurations. This is evidenced by observing the Q_{z} variation given that at the start of the meal, with low IOB coming from the basal infusion, the more aggressive controller according to vQ_{BPS} is able to react quickly to the disturbance. The same effect, i.e., with respect to aggressiveness of the controller when Q_{z} is a function of IOB, is similarly observed in situations not encompassing operability of the BPS, i.e., when comparing vQ versus cQ. As demonstrated, the additional operation of the BPS, as against configurations cQ and vQ, emphasizes insulin increase at the beginning of BG rise. Thus, as will be appreciated from the above, a controller of the AID system herein can, in response to detection of an elevation in one or more CGM measurements, initially increase basal insulin infusion to a subject to obtain a minimum amount of the IOB of the subject (IOBₘᵢₙ), in which IOBₘᵢₙ comprises a fraction of total daily insulin (TDI) required by the subject, and thereafter decrease the infusion according to a detuning or decrease of Q_{z}. Additionally, in response to detection of the elevation in one or more of the CGM measurements (arising from a glycemic disturbance such as an unannounced meal), such controller can supplement the initial infusion of IOBₘᵢₙ with a BPS insulin bolus responsive to the elevation to effect the real-time glycemia of the subject to be between the range of 70 mg/dL to 180 mg/dL.

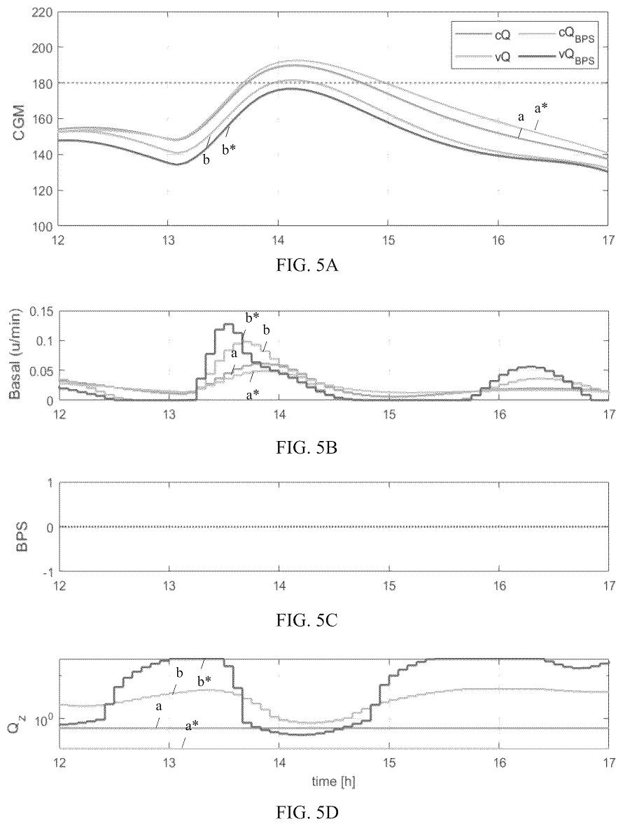

In referring to FIG. 4 illustrating glycemic outcome metrics including percentage of insulin infused during the first and second hours, PP_{H1} and PP_{H2}, respectively after lunch during the study, sections (a)-(d) correspond to configurations cQ, cQ_{BPS}, vQ, and vQ_{BPS}, respectively. When paired with the BPS, Q_{z}(IOB), i.e., configuration vQ_{BPS} corresponding to section b*, induces a proportion of 70/30 of insulin injected within the first and second hours of the postprandial state, respectively, relative to the difference in injected insulin being statistically significant at the 0.05 level (Wilcoxon signed rank test). It is worth highlighting that this difference is significantly higher than that of any of the other controller configurations (e.g., p<0.05 for each pair).

As demonstrated, greater TIR may be obtained for configuration vQ, implying better performance according Q_{z}(IOB), despite the fact that there is no statistically significant difference (p>0.05) between the distribution of the insulin infusion during the first and second hour for configurations cQ_{BPS} and vQ. This indicates that while BPS alone can increase the insulin infused as the disturbance is detected, the key factor increasing the achievable closed-loop performance is the shape of Q_{z} (i.e., whether Qz is dependent on IOB or not) and its capacity to detune the MPC controller in hyper-insulinemic situations.

### Scenario 2

In this second scenario, a focus was placed on variability in meal absorption. To this end, six meals with sizes of 0.75, 1, 0.9, 0.85, 0.7, and 1 g/kg were provided at 7:00 h, 12:00 h and 19:00 h each day. Breakfasts are considered fast-absorbing meals, while lunch and dinner are regarded as slow-absorbing meals. Additionally, two fast-absorbing snacks were provided at 11:00 h and 15:00 h each day, with sizes of 0.35, 0.4, 0.2, and 0.5 g/kg respectively. Intraday variability in insulin sensitivity and dawn phenomenon were also included.

Glycemic outcome metrics on this scenario for the 100-adult population are presented in Table 4. As provided, better performance (i.e., higher TIR and lower TAR) is obtained with controller configurations vQ and vQ_{BPS} than are obtained with controller configurations cQ and cQ_{BPS}, respectively. Moreover, considering a more challenging scenario with more meals and different meal absorption rates, no significant increase in TAR was observed between both scenarios.

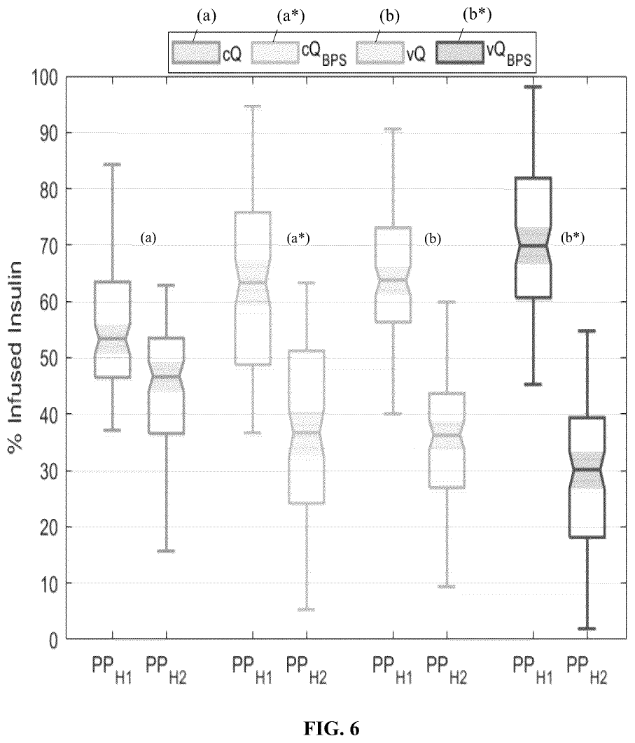

In referring to FIGS. 5(a)-5(d) illustrating the same metric types as to FIGS. 3(a)-3(d) and retaining similar reference to lines a, a*, b, and b*, the closed-loop postprandial excursion obtained with all configurations for a representative subject to lunch during the study is presented. This meal is selected considering it is a larger, slow-absorbing meal compared to the one analyzed in Scenario 1. In this case, the meal's lower absorption rate does not imply a sufficiently sharp blood glucose (BG) increase for the BPS to trigger a bolus, such that the only difference between cases is given by Q_{z}-scheduling. The same behavior as in Scenario 1 is observed, i.e., an increased insulin infusion toward the start of the meals with controller configurations vQ and vQ_{BPS} is obtained when compared to compared to the constant cases, i.e., controller configurations cQ and cQ_{BPS}. The main difference between the former cases can be attributed to the parameter selection, with vQ_{BPS} being more aggressive than is vQ.

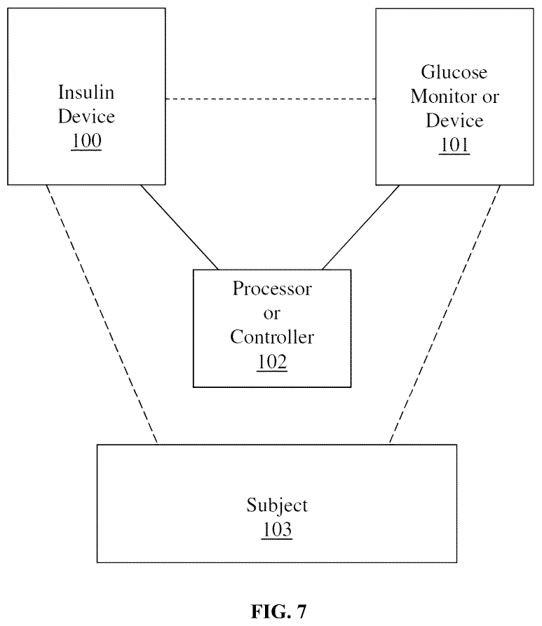

With the same section denotation applying to the controller configurations as are provided in FIG. 4, insulin distribution for the 100-adult cohort is presented in FIG. 6. The same phenomena, i.e., where the percentage insulin infused is significantly increased during the first hour is observed under this scenario. As will be appreciated, this situation is advantageous, also in the case of slow-absorbing meals, where mismatch in timing between meal and insulin absorption can be reduced.

In view of the above, adaptation of the Q_{z} error weight according to the MPC's cost function in favor of IOB, than when in favor of a constant Q_{z}, improves TIR in response to instance of unannounced meals through increasing the MPC's controller aggressiveness, e.g., through controller configurations vQ as well as vQ_{BPS}, when detecting lower concentrations of IOB. All the while, one or more of such controller configurations permits concentrating the insulin infusion during the first postprandial hour and at the same time avoiding insulin stacking.

In regard to the above, the AID system optionally embodied herein as the Rocket AP may include the MPC discussed herein as implemented in a Diabetes Assistant (DiA) format 20 provided by, for example, a smartphone or other receiving and/or computing platform configured to enable communication among an insulin infusion pump and a CGM and the DiA. As such, the DiA may define a general control paradigm and may be referred to herein as a “controller” tasked with continually predicting future glycemia values and calculating optimal insulin doses to maintain an individual's target glucose level. As such, one or more components of the aforementioned AID may be implemented by all appropriate software and/or hardware for carrying out operations referred to throughout discussions herein.

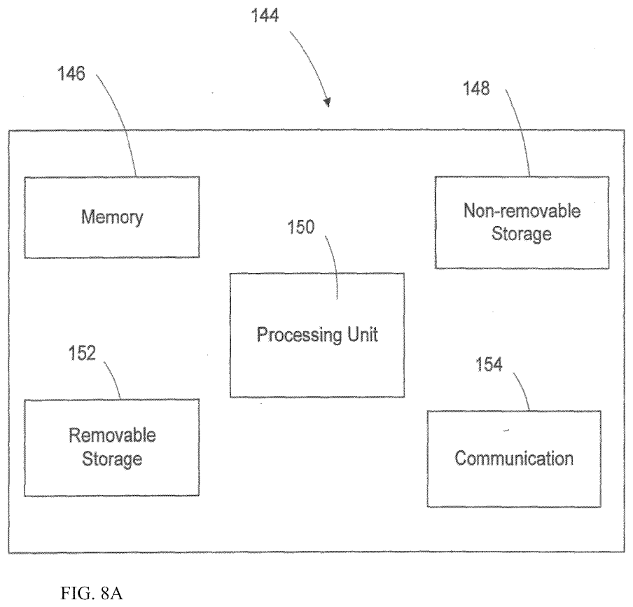

Thus, in referring to FIG. 7, a processor or controller 102, as embodied, for instance, by a DiA, communicates with the glucose monitor or device 101, and the insulin device 100. The processor or controller 102, as embodied by the DiA, may be configured to include all necessary hardware and/or software necessary to perform any and all required instructions, or portions thereof, to achieve the aforementioned tasks discussed herein, e.g., bolus calculation. The glucose monitor or device 101 communicates with the subject 103 to monitor glucose levels of the subject 103. The processor or controller 102 is configured to perform the required calculations. Optionally, the insulin device 100 communicates with the subject 103 to deliver insulin to the subject 103. The processor or controller 102 is configured to perform the required calculations. The glucose monitor 101 and the insulin device 100 may be implemented as a separate device or as a single device. The processor 102 may be implemented locally in the glucose monitor 101, the insulin device 100, or a standalone device (or in any combination of two or more of the glucose monitor, insulin device, or a standalone device). The processor 102 or a portion of the system may be located remotely such that the device is operated as a telemedicine device.

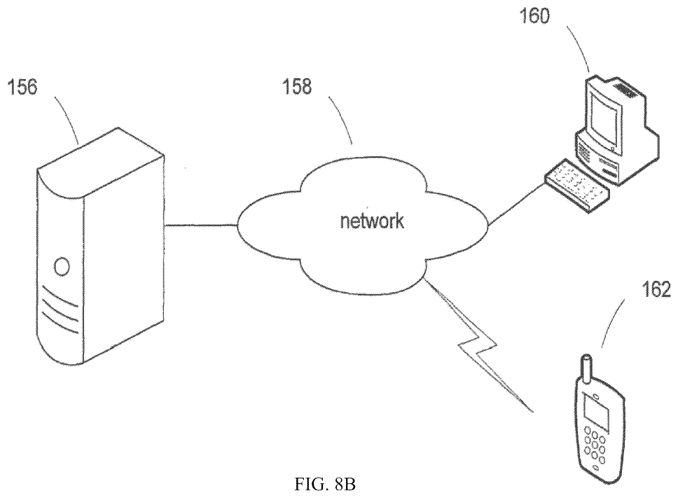

In referring to FIG. 8A, in its most basic configuration, computing device 144, optionally implementing the DiA, typically includes at least one processing unit 150 and memory 146. Depending on the exact configuration and type of computing device, memory 146 may be volatile (such as RAM), non-volatile (such as ROM, flash memory, etc.) or some combination of the two.

Additionally, device 144 may also have other features and/or functionality. For example, the device could also include additional removable and/or non-removable storage including, but not limited to, magnetic or optical disks or tape, as well as writable electrical storage media. Such additional storage is the figure by removable storage 152 and non-removable storage 148. Computer storage media includes volatile and nonvolatile, removable and non-removable media implemented in any method or technology for storage of information such as computer readable instructions, data structures, program modules or other data. The memory, the removable storage and the non-removable storage are all examples of computer storage media. Computer storage media includes, but is not limited to, RAM, ROM, EEPROM, flash memory or other memory technology CDROM, digital versatile disks (DVD) or other optical storage, magnetic cassettes, magnetic tape, magnetic disk storage or other magnetic storage devices, or any other medium which may be used to store the desired information and which may accessed by the device. Any such computer storage media may be part of, or used in conjunction with, the device.

The device may also contain one or more communications connections 154 that allow the device to communicate with other devices (e.g. other computing devices). The communications connections carry information in a communication media. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode, execute, or process information in the signal. By way of example, and not limitation, communication medium includes wired media such as a wired network or direct-wired connection, and wireless media such as radio, RF, infrared and other wireless media. As discussed above, the term computer readable media as used herein includes both storage media and communication media.

In referring to FIG. 8B, embodiments herein may also be implemented on a network system comprising a plurality of computing devices that are in communication with a networking means, such as a network with an infrastructure or an ad hoc network. The network connection may be wired connections or wireless connections. In this example, the network system comprises computer 156 (e.g. a network server), network connection means 158 (e.g. wired and/or wireless connections), computer terminal 160, and PDA (e.g. a smart-phone) 162 (or other handheld or portable device, such as a cell phone, laptop computer, tablet computer, GPS receiver, mp3 player, handheld video player, pocket projector, etc. or handheld devices (or non-portable devices) with combinations of such features). In an embodiment, it should be appreciated that the module listed as 156 may be glucose monitor device. In an embodiment, it should be appreciated that the module listed as 156 may be a glucose monitor device, artificial pancreas, and/or an insulin device (or other interventional or diagnostic device). Any of the components shown or discussed with FIG. 8B may be multiple in number. The embodiments herein may be implemented in anyone of the devices of the system. For example, execution of the instructions or other desired processing may be performed on the same computing device that is anyone of 156, 160, and 162. Alternatively, an embodiment may be performed on different computing devices of the network system. For example, certain desired or required processing or execution may be performed on one of the computing devices of the network (e.g. server 156 and/or glucose monitor device), whereas other processing and execution of the instruction may be performed at another computing device (e.g. terminal 160) of the network system, or vice versa. In fact, certain processing or execution may be performed at one computing device (e.g. server 156 and/or insulin device, AP, or glucose monitor device (or other interventional or diagnostic device)); and the other processing or execution of the instructions may be performed at different computing devices that may or may not be networked. For example, the certain processing may be performed at terminal 160, while the other processing or instructions are passed to device 162 where the instructions are executed. This scenario may be of particular value especially when the PDA 162 device, for example, accesses to the network through computer terminal 160 (or an access point in an ad hoc network). For another example, software to be protected may be executed, encoded or processed with one or more embodiments herein. The processed, encoded or executed software may then be distributed to customers. The distribution may be in a form of storage media (e.g. disk) or electronic copy.

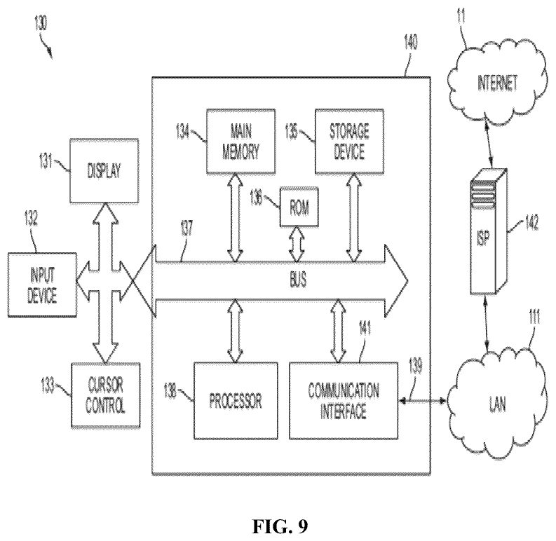

In referring to FIG. 9, there is shown a block diagram that illustrates a system 130 including a computer system 140 and the associated Internet 11 connection upon which an embodiment may be implemented. Such configuration is typically used for computers (hosts) connected to the Internet 11 and executing a server or a client (or a combination) software. A source computer such as laptop, an ultimate destination computer and relay servers, for example, as well as any computer or processor described herein, may use the computer system configuration and the Internet connection shown in FIG. 9. The system 140 may be used as a portable electronic device such as a notebook/laptop computer, a media player (e.g., MP3 based or video player), a cellular phone, a Personal Digital Assistant (PDA), a glucose monitor device, an artificial pancreas, an insulin delivery device (or other interventional or diagnostic device), an image processing device (e.g., a digital camera or video recorder), and/or any other handheld computing devices, or a combination of any of these devices. Note that while FIG. 9 illustrates various components of a computer system, it is not intended to represent any particular architecture or manner of interconnecting the components; as such details are not germane to the embodiments herein. It will also be appreciated that network computers, handheld computers, cell phones and other data processing systems which have fewer components or perhaps more components may also be used. The computer system of FIG. 9 may, for example, be an Apple Macintosh computer or Power Book, or an IBM compatible PC. Computer system 140 includes a bus 137, an interconnect, or other communication mechanism for communicating information, and a processor 138, commonly in the form of an integrated circuit, coupled with bus 137 for processing information and for executing the computer executable instructions. Computer system 140 also includes a main memory 134, such as a Random Access Memory (RAM) or other dynamic storage device, coupled to bus 137 for storing information and instructions to be executed by processor 138.

Main memory 134 also may be used for storing temporary variables or other intermediate information during execution of instructions to be executed by processor 138. Computer system 140 further includes a Read Only Memory (ROM) 136 (or other non-volatile memory) or other static storage device coupled to bus 137 for storing static information and instructions for processor 138. A storage device 135, such as a magnetic disk or optical disk, a hard disk drive for reading from and writing to a hard disk, a magnetic disk drive for reading from and writing to a magnetic disk, and/or an optical disk drive (such as DVD) for reading from and writing to a removable optical disk, is coupled to bus 137 for storing information and instructions. The hard disk drive, magnetic disk drive, and optical disk drive may be connected to the system bus by a hard disk drive interface, a magnetic disk drive interface, and an optical disk drive interface, respectively. The drives and their associated computer-readable media provide non-volatile storage of computer readable instructions, data structures, program modules and other data for the general purpose computing devices. Typically, computer system 140 includes an Operating System (OS) stored in a non-volatile storage for managing the computer resources and provides the applications and programs with an access to the computer resources and interfaces. An operating system commonly processes system data and user input, and responds by allocating and managing tasks and internal system resources, such as controlling and allocating memory, prioritizing system requests, controlling input and output devices, facilitating networking and managing files. Non-limiting examples of operating systems are Microsoft Windows, Mac OS X, and Linux.

The term “processor” is meant to include any integrated circuit or other electronic device (or collection of devices) capable of performing an operation on at least one instruction including, without limitation, Reduced Instruction Set Core (RISC) processors, CISC microprocessors, Microcontroller Units (MCUs), CISC-based Central Processing Units (CPUs), and Digital Signal Processors (DSPs). The hardware of such devices may be integrated onto a single substrate (e.g., silicon “die”), or distributed among two or more substrates. Furthermore, various functional aspects of the processor may be implemented solely as software or firmware associated with the processor.

Computer system 140 may be coupled via bus 137 to a display 131, such as a Cathode Ray Tube (CRT), a Liquid Crystal Display (LCD), a flat screen monitor, a touch screen monitor or similar means for displaying text and graphical data to a user. The display may be connected via a video adapter for supporting the display. The display allows a user to view, enter, and/or edit information that is relevant to the operation of the system. An input device 132, including alphanumeric and other keys, is coupled to bus 137 for communicating information and command selections to processor 138. Another type of user input device is cursor control 133, such as a mouse, a trackball, or cursor direction keys for communicating direction information and command selections to processor 138 and for controlling cursor movement on display 131. This input device typically has two degrees of freedom in two axes, a first axis (e.g., x) and a second axis (e.g., y), that allows the device to specify positions in a plane.

The computer system 140 may be used for implementing the methods and techniques described herein. According to one embodiment, those methods and techniques are performed by computer system 140 in response to processor 138 l executing one or more sequences of one or more instructions contained in main memory 134. Such instructions may be read into main memory 134 from another computer-readable medium, such as storage device 135. Execution of the sequences of instructions contained in main memory 134 causes processor 138 to perform the process steps described herein. In alternative embodiments, hard-wired circuitry may be used in place of or in combination with software instructions to implement the arrangement. Thus, embodiments herein are not limited to any specific combination of hardware circuitry and software.

The term “computer-readable medium” (or “machine-readable medium”) as used herein is an extensible term that refers to any medium or any memory, that participates in providing instructions to a processor, (such as processor 138) for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, and transmission medium. Transmission media includes coaxial cables, copper wire and fiber optics, including the wires that comprise bus 137. Transmission media may also take the form of acoustic or light waves, such as those generated during radio-wave and infrared data communications, or other form of propagated signals (e.g., carrier waves, infrared signals, digital signals, etc.). Common forms of computer-readable media include, for example, a floppy disk, a flexible disk, hard disk, magnetic tape, or any other magnetic medium, a CD-ROM, any other optical medium, punch-cards, paper-tape, any other physical medium with patterns of holes, a RAM, a PROM, and EPROM, a FLASH-EPROM, any other memory chip or cartridge, a carrier wave as described hereinafter, or any other medium from which a computer may read.

Various forms of computer-readable media may be involved in carrying one or more sequences of one or more instructions to processor 138 for execution. For example, the instructions may initially be carried on a magnetic disk of a remote computer. The remote computer may load the instructions into its dynamic memory and send the instructions over a telephone line using a modem. A modem local to computer system 140 may receive the data on the telephone line and use an infra-red transmitter to convert the data to an infra-red signal. An infra-red detector may receive the data carried in the infra-red signal and appropriate circuitry may place the data on bus 137. Bus 137 carries the data to main memory 134, from which processor 138 retrieves and executes the instructions. The instructions received by main memory 134 may optionally be stored on storage device 135 either before or after execution by processor 138.

Computer system 140 also includes a communication interface 141 coupled to bus 137. Communication interface 141 provides a two-way data communication coupling to a network link 139 that is connected to a local network 111. For example, communication interface 141 may be an Integrated Services Digital Network (ISDN) card or a modem to provide a data communication connection to a corresponding type of telephone line. As another non-limiting example, communication interface 141 may be a local area network (LAN) card to provide a data communication connection to a compatible LAN. For example, Ethernet based connection based on IEEE802.3 standard may be used such as 10/100BaseT, 1000BaseT (gigabit Ethernet), 10 gigabit Ethernet (10 GE or 10 GbE or 10 GigE per IEEE Std 802.3ae-2002 as standard), 40 Gigabit Ethernet (40 GbE), or 100 Gigabit Ethernet (100 GbE as per Ethernet standard IEEE P802.3ba), as described in Cisco Systems, Inc. Publication number 1-587005-001-3 (6/99), “Internetworking Technologies Handbook”, Chapter 7: “Ethernet Technologies”, pages 7-1 to 7-38, which is incorporated in its entirety for all purposes as if fully set forth herein. In such a case, the communication interface 141 typically include a LAN transceiver or a modem, such as Standard Microsystems Corporation (SMSC) LAN91C111 10/100 Ethernet transceiver described in the Standard Microsystems Corporation (SMSC) data-sheet “LAN91C111 10/100 Non-PCI Ethernet Single Chip MAC+PHY” Data-Sheet, Rev. 15 (02-20-04), which is incorporated in its entirety for all purposes as if fully set forth herein.

Wireless links may also be implemented. In any such implementation, communication interface 141 sends and receives electrical, electromagnetic or optical signals that carry digital data streams representing various types of information.

Network link 139 typically provides data communication through one or more networks to other data devices. For example, network link 139 may provide a connection through local network 111 to a host computer or to data equipment operated by an Internet Service Provider (ISP) 142. ISP 142 in turn provides data communication services through the worldwide packet data communication network Internet 11. Local network 111 and Internet 11 both use electrical, electromagnetic or optical signals that carry digital data streams. The signals through the various networks and the signals on the network link 139 and through the communication interface 141, which carry the digital data to and from computer system 140, are exemplary forms of carrier waves transporting the information.

A received code may be executed by processor 138 as it is received, and/or stored in storage device 135, or other non-volatile storage for later execution. In this manner, computer system 140 may obtain application code in the form of a carrier wave.

The concept of a personalized artificial pancreas system with an automatic BPS and enhanced safety by the present inventors. As seen from the algorithm and methodology requirements discussed herein, the procedure is readily applicable into devices, such as glucose devices, insulin devices, AP devices, and other interventional or diagnostic devices, and may be implemented and utilized with the related processors, networks, computer systems, internet, and components and functions according to the schemes disclosed herein.

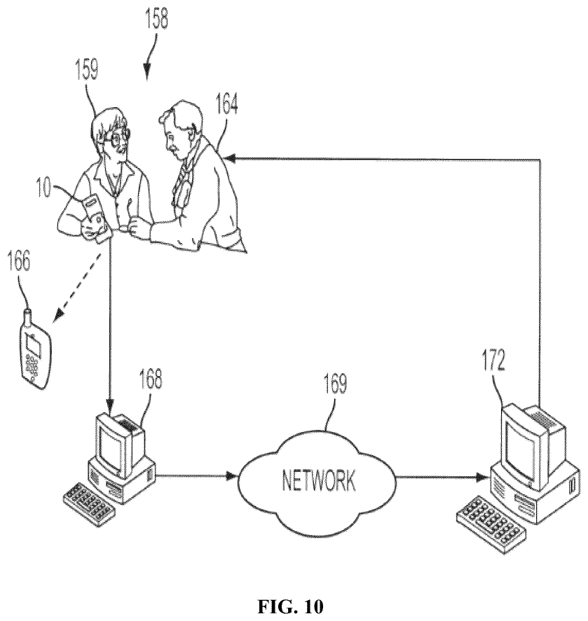

In referring to FIG. 10, there is illustrated a system in which one or more embodiments herein may be implemented using a network, or portions of a network or computers, although the presently discussed glucose monitor, AP or insulin device (or other interventional or diagnostic device) may be practiced without a network.

FIG. 10 diagrammatically illustrates an exemplary system in which examples of the embodiments herein may be implemented. In an embodiment the glucose monitor, AP or insulin device (or other interventional or diagnostic device) may be implemented by the subject (or patient) locally at home or other desired location. However, in an alternative embodiment it may be implemented in a clinic setting or assistance setting. For instance, referring to FIG. 14, a clinic setup 158 provides a place for doctors (e.g. 164) or clinician/assistant to diagnose patients (e.g. 159) with diseases related with glucose and related diseases and conditions. A glucose monitoring device 10 may be used to monitor and/or test the glucose levels of the patient—as a standalone device. It should be appreciated that while only glucose monitor device 10 is shown in the figure, the system of the embodiments herein and any component thereof may be used in the manner depicted by FIG. 10. The system or component may be affixed to the patient or in communication with the patient as desired or required. For example the system or combination of components thereof—including a glucose monitor device 10 (or other related devices or systems such as a controller, and/or an artificial pancreas, an insulin pump (or other interventional or diagnostic device), or any other desired or required devices or components)—may be in contact, communication or affixed to the patient through tape or tubing (or other medical instruments or components) or may be in communication through wired or wireless connections. Such monitor and/or test may be short term (e.g. clinical visit) or long term (e.g. clinical stay or family). The glucose monitoring device outputs may be used by the doctor (clinician or assistant) for appropriate actions, such as insulin injection or food feeding for the patient, or other appropriate actions or modeling. Alternatively, the glucose monitoring device output may be delivered to computer terminal 168 for instant or future analyses. The delivery may be through cable or wireless or any other suitable medium. The glucose monitoring device output from the patient may also be delivered to a portable device, such as PDA 166. The glucose monitoring device outputs with improved accuracy may be delivered to a glucose monitoring center 172 for processing and/or analyzing. Such delivery may be accomplished in many ways, such as network connection 169, which may be wired or wireless.

In addition to the glucose monitoring device outputs, errors, parameters for accuracy improvements, and any accuracy related information may be delivered, such as to computer 168, and/or glucose monitoring center 172 for performing error analyses. This may provide a centralized accuracy monitoring, modeling and/or accuracy enhancement for glucose centers, due to the importance of the glucose sensors.

Examples of the embodiments herein may also be implemented in a standalone computing device associated with the target glucose monitoring device, artificial pancreas, and/or insulin device (or other interventional or diagnostic device). An exemplary computing device (or portions thereof) in which examples of the embodiments herein may be implemented is schematically illustrated in FIG. 8A.

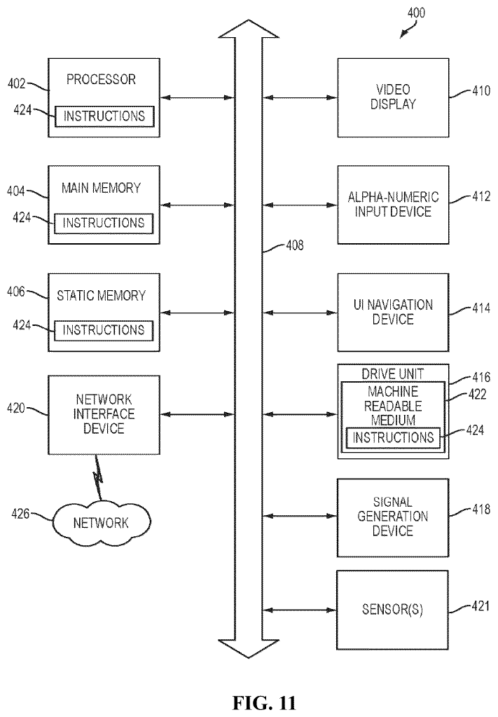

In referring to FIG. 11, there is shown a block diagram illustrating an example of a machine upon which one or more aspects of embodiments herein may be implemented.

FIG. 11 illustrates a block diagram of an example machine 400 upon which one or more embodiments (e.g., discussed methodologies) may be implemented (e.g., run).

Examples of machine 400 may include logic, one or more components, circuits (e.g., modules), or mechanisms. Circuits are tangible entities configured to perform certain operations. In an example, circuits may be arranged (e.g., internally or with respect to external entities such as other circuits) in a specified manner. In an example, one or more computer systems (e.g., a standalone, client or server computer system) or one or more hardware processors (processors) may be configured by software (e.g., instructions, an application portion, or an application) as a circuit that operates to perform certain operations as described herein. In an example, the software may reside (1) on a non-transitory machine readable medium or (2) in a transmission signal. In an example, the software, when executed by the underlying hardware of the circuit, causes the circuit to perform the certain operations.

In an example, a circuit may be implemented mechanically or electronically. For example, a circuit may comprise dedicated circuitry or logic that is specifically configured to perform one or more techniques such as discussed above, such as including a special-purpose processor, a field programmable gate array (FPGA) or an application-specific integrated circuit (ASIC). In an example, a circuit may comprise programmable logic (e.g., circuitry, as encompassed within a general-purpose processor or other programmable processor) that may be temporarily configured (e.g., by software) to perform the certain operations. It will be appreciated that the decision to implement a circuit mechanically (e.g., in dedicated and permanently configured circuitry), or in temporarily configured circuitry (e.g., configured by software) may be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor may be configured as respective different circuits at different times. Software may accordingly configure a processor, for example, to constitute a particular circuit at one instance of time and to constitute a different circuit at a different instance of time.

In an example, circuits may provide information to, and receive information from, other circuits. In this example, the circuits may be regarded as being communicatively coupled to one or more other circuits. Where multiple of such circuits exist contemporaneously, communications may be achieved through signal transmission (e.g., over appropriate circuits and buses) that connect the circuits. In embodiments in which multiple circuits are configured or instantiated at different times, communications between such circuits may be achieved, for example, through the storage and retrieval of information in memory structures to which the multiple circuits have access. For example, one circuit may perform an operation and store the output of that operation in a memory device to which it is communicatively coupled. A further circuit may then, at a later time, access the memory device to retrieve and process the stored output. In an example, circuits may be configured to initiate or receive communications with input or output devices and may operate on a resource (e.g., a collection of information).

The various operations of method examples described herein may be performed, at least partially, by one or more processors that are temporarily configured (e.g., by software) or permanently configured to perform the relevant operations. Whether temporarily or permanently configured, such processors may constitute processor-implemented circuits that operate to perform one or more operations or functions. In an example, the circuits referred to herein may comprise processor-implemented circuits.

Similarly, the methods described herein may be at least partially processor-implemented. For example, at least some of the operations of a method may be performed by one or processors or processor-implemented circuits. The performance of certain of the operations may be distributed among the one or more processors, not only residing within a single machine, but deployed across a number of machines. In an example, the processor or processors may be located in a single location (e.g., within a home environment, an office environment or as a server farm), while in other examples the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) may be implemented in digital electronic circuitry, in computer hardware, in firmware, in software, or in any combination thereof. Example embodiments may be implemented using a computer program product (e.g., a computer program, tangibly embodied in an information carrier or in a machine readable medium, for execution by, or to control the operation of, data processing apparatus such as a programmable processor, a computer, or multiple computers).

A computer program may be written in any form of programming language, including compiled or interpreted languages, and it may be deployed in any form, including as a stand-alone program or as a software module, subroutine, or other unit suitable for use in a computing environment. A computer program may be deployed to be executed on one computer or on multiple computers at one site or distributed across multiple sites and interconnected by a communication network.

In an example, operations may be performed by one or more programmable processors executing a computer program to perform functions by operating on input data and generating output. Examples of method operations may also be performed by, and example apparatus may be implemented as, special purpose logic circuitry (e.g., a field programmable gate array (FPGA) or an application-specific integrated circuit (ASIC)).

The computing system may include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice. Below are set out hardware (e.g., machine 400) and software architectures that may be deployed in example embodiments.

In an example, the machine 400 may operate as a standalone device or the machine 400 may be connected (e.g., networked) to other machines.

In a networked deployment, the machine 400 may operate in the capacity of either a server or a client machine in server-client network environments. In an example, machine 400 may act as a peer machine in peer-to-peer (or other distributed) network environments. The machine 400 may be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 may include a processor 402 (e.g., a central processing unit (CPU), a graphics processing unit (GPU) or both), a main memory 404 and a static memory 406, some or all of which may communicate with each other via a bus 408. The machine 400 may further include a display unit 410, an alphanumeric input device 412 (e.g., a keyboard), and a user interface (UI) navigation device 411 (e.g., a mouse). In an example, the display unit 410, input device 412 and UI navigation device 414 may be a touch screen display. The machine 400 may additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 may include a machine readable medium 422 on which is stored one or more sets of data structures or instructions 424 (e.g., software) embodying or utilized by any one or more of the methodologies or functions described herein. The instructions 424 may also reside, completely or at least partially, within the main memory 404, within static memory 406, or within the processor 402 during execution thereof by the machine 400. In an example, one or any combination of the processor 402, the main memory 404, the static memory 406, or the storage device 416 may constitute machine readable media.

While the machine readable medium 422 is illustrated as a single medium, the term “machine readable medium” may include a single medium or multiple media (e.g., a centralized or distributed database, and/or associated caches and servers) that configured to store the one or more instructions 424. The term “machine readable medium” may also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” may accordingly be taken to include, but not be limited to, solid-state memories, and optical and magnetic media. Specific examples of machine readable media may include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 may further be transmitted or received over a communications network 426 using a transmission medium via the network interface device 420 utilizing any one of a number of transfer protocols (e.g., frame relay, IP, TCP, UDP, HTTP, etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

As discussed herein, a “subject” may be any applicable human, animal, or other organism, living or dead, or other biological or molecular structure or chemical environment, and may relate to particular components of the subject, for instance specific tissues or fluids of a subject (e.g., human tissue in a particular area of the body of a living subject), which may be in a particular location of the subject.

While the present disclosure has been described with respect to specific embodiments, many modifications, variations, alterations, substitutions, and equivalents will be apparent to those skilled in the art. The present disclosure is not to be limited in scope by the specific embodiment described herein. Indeed, various modifications of the embodiments herein, in addition to those described herein, will be apparent to those of skill in the art from the foregoing description and accompanying drawings. Accordingly, the embodiments herein are to be considered as limited only by the spirit and scope of the disclosure (and claims), including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity may be repeated, any activity may be performed by multiple entities, and/or any element may be duplicated. Further, any activity or element may be excluded, the sequence of activities may vary, and/or the interrelationship of elements may vary. Unless clearly specified to the contrary, there is no requirement for any particular described or illustrated activity or element, any particular sequence or such activities, any particular size, speed, material, dimension or frequency, or any particular interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

Where applicable, citations herein, whether by numerical indication or by other means, refer to one or more of the documents listed in the section entitled “References.”

### REFERENCES

The devices, systems, apparatuses, modules, compositions, computer program products, non-transitory computer readable medium, models, algorithms, and methods of various embodiments disclosed herein may utilize aspects (devices, systems, apparatuses, modules, compositions, computer program products, non-transitory computer readable medium, models, algorithms, and methods) disclosed in the following references, applications, publications and patents and which are hereby incorporated by reference herein in their entirety, and which are not admitted to be prior art with respect to the present embodiments by inclusion in this section:
Boiroux, D., Aradóttir, T.B., Nørgaard, K., Poulsen, N.K., Madsen, H., and Jørgensen, J.B. (2017). An adaptive nonlinear basal-bolus calculator for patients with type 1 diabetes. Journal of Diabetes Science and Technology, 11(1), 29-36.
Camacho, E.F. and Alba, C.B. (2013). Model predictive control. Springer Science & Business Media.
Colmegna, P., Cengiz, E., Garcia-Tirado, J., Kraemer, K., and Breton, M.D. (2021). Impact of accelerating insulin on an artificial pancreas system without meal announcement: an in silico examination. Journal of Diabetes Science and Technology, 15(4), 833-841.
Garcia-Tirado, J., Colmegna, P., Corbett, J.P., Ozaslan, B., and Breton, M.D. (2019). In silico analysis of an exercise-safe artificial pancreas with multistage model predictive control and insulin safety system. Journal of Diabetes Science and Technology, 13(6), 1054-1064.
Garcia-Tirado, J., Lv, D., Corbett, J.P., Colmegna, P., and Breton, M.D. (2021). Advanced hybrid artificial pan- creas system improves on unannounced meal response-in silico comparison to currently available system. Computer Methods and Programs in Biomedicine, 211, 106401.
Gondhalekar, R., Dassau, E., and Doyle III, F.J. (2018). Velocity-weighting & velocity-penalty mpc of an artificial pancreas: Improved safety & performance. Automatica, 91, 105-117.
Hajizadeh, I., Samadi, S., Sevil, M., Rashid, M., and Cinar, A. (2019). Performance assessment and modification of an adaptive model predictive control for automated insulin delivery by a multivariable artificial pancreas. Industrial and Engineering Chemistry Research, 58(26), 11506-11520.
Hovorka, R., Canonico, V., Chassin, L.J., Haueter, U., Massi-Benedetti, M., Federici, M.O., Pieber, T.R., Schaller, H.C., Schaupp, L., Vering, T., et al. (2004). Nonlinear model predictive control of glucose concentration in subjects with type 1 diabetes. Physiological measurement, 25(4), 905.
Kovatchev, B.P., Breton, M., Man, C.D., and Cobelli, C. (2009). In silico preclinical trials: A proof of concept in closed-loop control of type 1 diabetes. Journal of Diabetes Science and Technology, 3(1), 44-55.
Kovatchev, B.P., Cox, D.J., Gonder-Frederick, L.A., and Clarke, W. (1997). Symmetrization of the blood glucose measurement scale and its applications. Diabetes care, 20(11), 1655-1658.
Messori, M., Toffanin, C., Favero, S.D., De Nicolao, G., Cobelli, C., and Magni, L. (2016). Model individualization for artificial pancreas. Comput. Meth. Prog. Bio., (2016). DOI: 10.1016/j.cmpb.2016.06.006. doi: 10.1016/j.cmpb.2016.06.006.
Swan, K.L., Dziura, J.D., Steil, G.M., Voskanyan, G.R., Sikes, K.A., Steffen, A.T., Martin, M.L., Tamborlane, W.V., and Weinzimer, S.A. (2009). Effect of age of infusion site and type of rapid-acting analog on pharmacodynamic parameters of insulin boluses in youth with type 1 diabetes receiving insulin pump therapy. Diabetes Care, 32(2), 240-244.
Villa-Tamayo, M.F. and Rivadeneira, P. (2020). Adaptive impulsive offset-free mpc to handle parameter variations for type 1 diabetes treatment. Industrial & Engineering Chemistry Research, 59(13), 5865-5876. doi: 10.1021/acs.iecr.9b05979.
Visentin, R., Campos-Náñez, E., Schiavon, M., Lv, D., Vettoretti, M., Breton, M., Kovatchev, B.P., Man, C.D., and Cobelli, C. (2018). The uva/padova type 1 diabetes simulator goes from single meal to single day. Journal of Diabetes Science and Technology, 12(2), 273-281.
Wolfsdorf, J.I. and Ratner, R.E. (2019). Sglt inhibitors for type 1 diabetes: proceed with extreme caution. Diabetes Care, 42(6), 991-993.

What is claimed is:

1. In an automated insulin delivery (AID) system, a processor-implemented method of regulating glycemia for a subject having Type 1 diabetes (T1D), comprising:
predicting blood glucose (BG) levels for the subject based on operation of a model predictive control (MPC) regime on continuous glucose monitor (CGM) measurements of the subject,
wherein the MPC regime weights a glucose target error thereof according to a predetermined weighting factor Q_{z} in dependence on insulin on board (IOB) of the subject; and
delivering basal insulin dosing to the subject according to the predicting to maintain real-time glycemia of the subject with a range of 70 mg/dL to 180 mg/dL.

2. The method of claim 1, wherein:
for the weighted glucose target error, Q_{z} weights a difference between the predicted BG levels for the subject and a reference BG level corresponding to the MPC regime.

3. The method of claim 1, wherein:
Q_{z} is adjusted based at least on a minimum amount of the IOB of the subject (IOBₘᵢₙ) in which IOBₘᵢₙ comprises a fraction of total daily insulin (TDI) required by the subject before Q_{z} can be decreased.

4. The method of claim 1, wherein:
in response to detection of an elevation in one or more of the CGM measurements, a controller of the AID system increases basal insulin infusion to the subject to obtain a minimum amount of the IOB of the subject (IOBₘᵢₙ) in which IOBₘᵢₙ comprises a fraction of total daily insulin (TDI) required by the subject before Q_{z} can be decreased.

5. The method of claim 4, wherein:
in response to detection of the elevation in one or more of the CGM measurements, the controller of the AID system supplements the IOBₘᵢₙ with an insulin bolus responsive to the elevation to effect the real-time glycemia of the subject to be between the range of 70 mg/dL to 180 mg/dL.

6. The method of claim 5, wherein:
the insulin bolus is measured according to a percentage of the TDI (P(TDI), based on a predetermined probability (πₖ) that the elevation in one or more of the CGM measurements resulted from a glycemic disturbance comprising at least an unannounced meal.

7. The method of claim 6, wherein:
the P(TDI) is given by the following schedule, in which:
$$P ( TDI ) = \{ \begin{matrix}0 \% if {π}_{k} < 0 \% \\ 7 \% if {π}_{k} \geq 30 \% \\ 10 \% if {π}_{k} \geq 40 \%\end{matrix} .$$

8. The method of claim 6, wherein:
as the IOB of the subject decreases, Q_{z} is increased to cause the controller of the AID system to increase the basal insulin infusion to the subject to obtain the real-time glycemia of the subject to be between the range of 70 mg/dL to 180 mg/dL.

9. An automated insulin delivery (AID) system regulating glycemia for a subject having Type 1 diabetes (T1D), comprising:
one or more processors;
one or more a processor-readable memories comprising processor-executable instructions for:
predicting blood glucose (BG) levels for the subject based on operation of a model predictive control (MPC) regime on continuous glucose monitor (CGM) measurements of the subject,
wherein the MPC regime weights a glucose target error thereof according to a predetermined weighting factor Q_{z} in dependence on insulin on board (IOB) of the subject; and
delivering basal insulin dosing to the subject according to the predicting to maintain the real-time glycemia of the subject with a range of 70 mg/dL to 180 mg/dL.

10. The system of claim 9, wherein:
for the weighted glucose target error, Q_{z} weights a difference between the predicted BG levels for the subject and a reference BG level corresponding to the MPC regime.

11. The system of claim 9, wherein:
Q_{z} is adjusted based at least on a minimum amount of the IOB of the subject (IOBₘᵢₙ) in which IOBₘᵢₙ comprises a fraction of total daily insulin (TDI) required by the subject before Q_{z} can be decreased.

12. The system of claim 9, wherein:
in response to detection of an elevation in one or more of the CGM measurements, a controller of the AID system increases basal insulin infusion to the subject to obtain a minimum amount of the IOB of the subject (IOBₘᵢₙ) in which IOBₘᵢₙ comprises a fraction of total daily insulin (TDI) required by the subject before Q_{z} can be decreased.

13. The system of claim 12, wherein:
in response to detection of the elevation in one or more of the CGM measurements, the controller of the AID system supplements the IOBₘᵢₙ with an insulin bolus responsive to the elevation to effect the real-time glycemia of the subject to be between the range of 70 mg/dL to 180 mg/dL.

14. The system of claim 13, wherein:
the insulin bolus is measured according to a percentage of the TDI (P(TDI), based on a predetermined probability (πₖ) that the elevation in one or more of the CGM measurements resulted from a glycemic disturbance comprising at least an unannounced meal.

15. The system of claim 14, wherein:
the P(TDI) is given by the following schedule, in which:
$$P ( TDI ) = \{ \begin{matrix}0 \% if {π}_{k} < 0 \% \\ 7 \% if {π}_{k} \geq 30 \% \\ 10 \% if {π}_{k} \geq 40 \%\end{matrix} .$$

16. The system of claim 15, wherein:
as the IOB of the subject decreases, Q_{z} is increased to cause the controller of the AID system to increase the basal insulin infusion to the subject to obtain the real-time glycemia of the subject to be between the range of 70 mg/dL to 180 mg/dL.

17. A non-transient computer-readable medium having stored thereon computer-executable instructions for regulating, by an automated insulin delivery (AID) system, glycemia for a subject having Type 1 diabetes (T1D), said instructions causing a computer to:
predict blood glucose (BG) levels for the subject based on operation of a model predictive control (MPC) regime on continuous glucose monitor (CGM) measurements of the subject,
wherein the MPC regime weights a glucose target error thereof according to a predetermined weighting factor Q_{z} in dependence on insulin on board (IOB) of the subject; and
deliver basal insulin dosing to the subject according to the predicting to maintain the real-time glycemia of the subject with a range of 70 mg/dL to 180 mg/dL.

18. The medium of claim 17, wherein:
for the weighted glucose target error, Q_{z} weights a difference between the predicted BG levels for the subject and a reference BG level corresponding to the MPC regime.

19. The medium of claim 17, wherein:
Q_{z} is adjusted based at least on a minimum amount of the IOB of the subject (IOBₘᵢₙ) in which IOBₘᵢₙ comprises a fraction of total daily insulin (TDI) required by the subject before Q_{z} can be decreased.

20. The medium of claim 17, wherein:
in response to detection of an elevation in one or more of the CGM measurements, a controller of the AID system increases basal insulin infusion to the subject to obtain a minimum amount of the IOB of the subject (IOBₘᵢₙ) in which IOBₘᵢₙ comprises a fraction of total daily insulin (TDI) required by the subject before Q_{z} can be decreased.

21. The medium of claim 20, wherein:
in response to detection of the elevation in one or more of the CGM measurements, the controller of the AID system supplements the IOBₘᵢₙ with an insulin bolus responsive to the elevation to effect the real-time glycemia of the subject to be between the range of 70 mg/dL to 180 mg/dL.

22. The medium of claim 21, wherein:
the insulin bolus is measured according to a percentage of the TDI (P(TDI), based on a predetermined probability (πₖ) that the elevation in one or more of the CGM measurements resulted from a glycemic disturbance comprising at least an unannounced meal.

23. The medium of claim 22, wherein:
the P(TDI) is given by the following schedule, in which:
$$P ( TDI ) = \{ \begin{matrix}0 \% if {π}_{k} < 0 \% \\ 7 \% if {π}_{k} \geq 30 \% \\ 10 \% if {π}_{k} \geq 40 \%\end{matrix} .$$

24. The medium of claim 23, wherein:
as the IOB of the subject decreases, Q_{z} is increased to cause the controller of the AID system to increase the basal insulin infusion to the subject to obtain the real-time glycemia of the subject to be between the range of 70 mg/dL to 180 mg/dL.
